(19) United States
(12) Patent Application Publication
(10) Pub. No.: US 2026/0021112 A1
(43) Pub. Date: Jan. 22, 2026

(54) DEXAMETHASONE FOR TREATING AUTISM

(71) Applicant: HAMAD BIN KHALIFA UNIVERSITY, Doha (QA)

(72) Inventors: Yongsoo Park, Doha (QA); Julie Valerie Caroline M Decock, Doha (QA)

(21) Appl. No.: 19/272,498

(22) Filed: Jul. 17, 2025

Related U.S. Application Data
(60) Provisional application No. 63/673,495, filed on Jul. 19, 2024.

(51) Int. Cl. A61K 31/573; A61P 25/00; G01N 33/68

(52) U.S. Cl. CPC A61K 31/573; A61P 25/00; G01N 33/6866; G01N 33/6896; G01N 2333/57; G01N 2800/30

(57) ABSTRACT

Described herein are methods for diagnosis, screening, and treatment of autism spectrum disorder. Specifically, the invention describes methods of treating autism using dexamethasone to inhibit extracellular vesicle (EV)-mediated IFN-gamma secretion and chronic inflammation. The invention further describes methods of identifying autism in a patient by measuring EV-mediated IFN-gamma secretion in patient samples.

### CROSS REFERENCE TO RELATED APPLICATIONS

This disclosure claims priority to U.S. Provisional Patent Application No. 63/673,495, filed Jul. 19, 2024, the entire contents of which are hereby incorporated by reference.

### TECHNICAL FIELD

Described herein are methods of identifying patients with autism and/or methods of using dexamethasone for treatment of autism.

### BACKGROUND

Autism spectrum disorder (ASD) manifests in early childhood and is characterized by qualitative abnormalities in social interactions, aberrant communication skills, and restricted repetitive behaviors, interests, and activities. Current treatments for ASD seek to reduce symptoms that interfere with daily functioning and quality of life. The causes of ASD are unknown, but hypotheses include obstetric complications, infection, genetics, and toxic exposures. There is a need to understand the etiology of ASD and methods of ASD treatment.

### SUMMARY

The present disclosure provides methods for autism spectrum disorder (ASD) diagnosis, screening, and/or treatment. In some embodiments, methods include identifying a subject with ASD. In other embodiments, the methods include treating a subject having ASD with dexamethasone, which can inhibit chronic inflammation.

Provided herein are methods of identifying a subject as having ASD comprising identifying an elevated interferon (IFN)-gamma secretion from the subject, wherein the elevated IFN-gamma secretion is from peripheral blood lymphocytes cells contacted with isolated extracellular vesicles.

Also provided herein are methods of treating ASD in a subject, a method comprising administering a therapeutically effective amount of dexamethasone to the subject.

### BRIEF DESCRIPTION OF THE DRAWINGS

FIG. 1 is a schematic diagram of IFN-gamma ELISpot assay. The activation and IFN-gamma secretion from peripheral blood lymphocytes (PBL) in response to extracellular vesicles (EVs) were measured using the IFN-gamma ELISpot assay. EVs were used intact (without lysis), and IFN-gamma antibody detected IFN-gamma released by PBL cells.

FIG. 2 provides representative images and quantifications of the IFN-gamma ELISpot assay. No signals were observed with healthy control (HC) and autism spectrum disorder (ASD) EVs alone and upon addition of peripheral blood lymphocytes (PBL), IFN-gamma spot forming units (SFU) were observed. ASD EVs increase the number of IFN-gamma SFU. The ratio of PBL to EVs is 1:100 and each experiment was performed in duplicate. Data are means±SD, with statistical analysis performed using an unpaired two-tailed t-test (HC EVs: n=11; ASD EVs: n=11).

FIG. 3 provides representative images and quantifications of the IFN-gamma ELISpot assay which demonstrate the inhibitory effect of dexamethasone (Dex) on ASD EV-induced PBL activation. PBL were incubated with either no EVs or ASD EVs in the absence or presence of Dex. Addition of Dex at 100 nM reduces ASD EV-mediated IFN-gamma secretion. Data are means±SD and unpaired two-tailed t-test was used (No Dex: n=9; Dex: n=9).

FIG. 4 provides quantifications of the IFN-gamma ELISpot assay which demonstrate that Dex had no significant impact on basal IFN-γ secretion from PBLs alone in the absence of EV stimulation. Data are presented as a percentage of control (No Dex, n=4).

### DETAILED DESCRIPTION OF THE INVENTION

Described herein are mechanisms related to the etiology of autism spectrum disorder (ASD). Provided herein are methods of treating ASD by reducing proinflammatory cytokines that are released by ASD extracellular vesicles (EVs).

Extracellular vesicles are secreted membrane-bound vesicles which mediate intercellular communication by transferring biomolecules such as proteins. Extracellular vesicles are secreted into the extracellular space by all cell types and can be found in bodily fluids (e.g. plasma, saliva, semen, urine, cerebral spinal fluid). They can be a source of circulating biomarkers for use as clinical diagnostics.

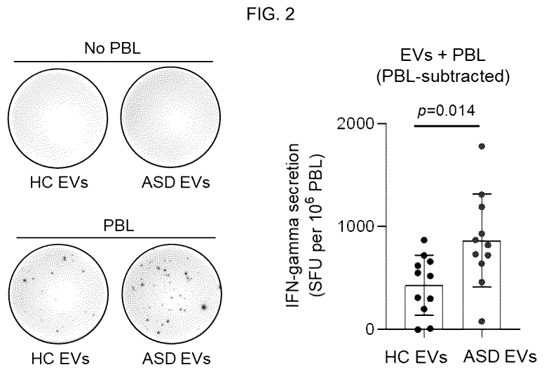

Isolated extracellular vesicles from autism spectrum disorder patients (collectively referred to herein as “ASD EVs”) can contain dysregulated cytokines and induce a proinflammatory response. In some embodiments, stimulating peripheral blood lymphocytes (PBL) with ASD EVs can induce secretion of elevated levels of the pro-inflammatory cytokine, interferon (IFN)-gamma (FIG. 2). This suggests that ASD EVs potentiate inflammatory response by increasing IFN-gamma secretion.

Inflammatory molecules, such as cytokines, have been implicated in influencing the developing brain. Elevated levels of specific cytokines can result in an inflammatory response in the brain, potentially disrupting normal developmental processes. This chronic inflammation and immune dysregulation have been proposed as potential contributors to the characteristic features of autism.

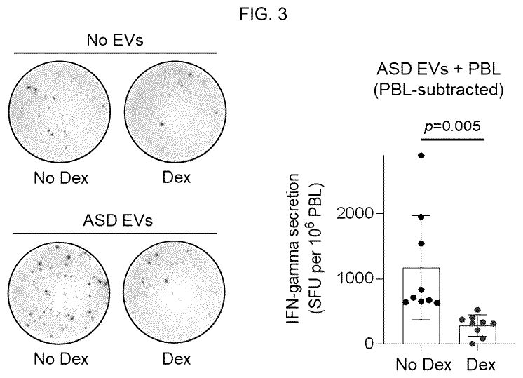

Given that ASD EVs promote an inflammatory response by increasing IFN-gamma secretion, dexamethasone, an anti-inflammatory medication can be used to treat ASD. In some embodiments, dexamethasone can effectively reduce IFN-gamma secretion triggered by ASD EVs (FIG. 3), demonstrating that dexamethasone can be therapeutic for ASD, such as through inhibiting chronic inflammation and mitigating associated ASD symptoms.

Provided herein is a method of identifying or diagnosing a subject as having ASD comprising: isolating extracellular vesicles from the subject, identifying if the subject has elevated IFN-gamma secretion from peripheral blood lymphocytes cells contacted with the isolated extracellular vesicles, and/or identifying the subject as having ASD if the IFN-gamma secretion is higher than when compared to the IFN-gamma secretion in healthy control subjects.

IFN-gamma secretion is considered elevated when the IFN-gamma secretion is higher than when compared to the IFN-gamma secretion in healthy control subjects or a reference level and/or threshold deriving thereof.

In some embodiments, a higher IFN-gamma secretion in ASD subjects when compared to healthy control subjects, can be, but not limited to, an increase of at least 5% as compared to a reference level, for example an increase of at least about 10%, or at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

In some embodiments, a higher IFN-gamma secretion in ASD subjects when compared to healthy control subjects, can be, but not limited to, an increase greater than one standard deviation as compared to a reference level. For example, an increase of two standard deviations above the mean of healthy control subjects would identify a patient as having ASD.

In some embodiments, the subject is identified as having ASD if the IFN-gamma secretion is greater than a threshold of one standard deviation above the mean of IFN-gamma secretion values from a cohort of healthy subjects.

In some embodiments, the subject is a developing fetus in utero and the subject is identified as having a susceptibility to developing ASD if the IFN-gamma secretion is higher when compared to the IFN-gamma secretion in healthy control subjects. Methods herein can be used in a prenatal diagnostic context.

In some embodiments, the methods described herein are used to identify or predict susceptibility to developing autism.

In some embodiments, the methods described herein are used to identify or diagnose a pediatric patient as having ASD.

In some embodiments, EVs are isolated using methods known in the art, including but not limited to, size exclusion chromatography, differential centrifugation, sucrose gradient centrifugation, microfiltration, pull-down with antibody-coated magnetic beads, microfluidic devices, and commercially available kits.

In some embodiments, IFN-gamma is detected through quantitative protein methods known in the art, including but not limited to, western blot, enzyme-linked immunosorbent assay (ELISA), enzyme-linked immunospot (ELISpot) assay, and commercially available kits.

In some embodiments, the patient/subject is a mammal. A mammal can be a human.

Provided herein are methods of treating ASD by administering to the subject a therapeutically effective amount of dexamethasone.

When administered to a human, dexamethasone is administered as a pharmaceutical composition comprising, for example, dexamethasone and a pharmaceutically acceptable carrier.

Therapeutically effective amounts of dexamethasone can range from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. In other embodiments, the dosage is 1 mg-500 mg. In some embodiments, the dosage is 10, 20, 30, 40, 50, 60, 70, 80, 90, 100, 110, 120, 130, 140, or 150 mg.

These doses can be unitary or divided and can be administered one or more times per day. The above dosages are exemplary of the average case, but there can be individual instances in which higher or lower dosages are merited, and such are within the scope of this disclosure. In practice, the physician determines therapeutically effective amounts and the actual dosing regimen that is most suitable for an individual subject, which can vary with the age, weight, and response of the particular subject.

In some embodiments, dexamethasone can be administered at a dosage of about 0.5 mg, 1 mg, 5 mg, 10 mg, 20 mg, 25 mg, 30 mg, 35 mg, 40 mg, 45 mg, or 50 mg.

Dexamethasone can be given per se or as a pharmaceutical composition containing, for example, 0.1 to 99.5% (or 0.5 to 90%) of the active ingredient in combination with a pharmaceutically-acceptable carrier.

### Definitions

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

“Therapeutically effective amount” or “effective amount” refer to that amount of dexamethasone and/or a pharmaceutical composition thereof that produces a beneficial result and is effective to elicit the desired biological or medical response when administered to a subject for treating a disease, is sufficient to affect such treatment for the disease. The effective amount can vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved.

“Administration” or “administering as used herein includes a single therapeutic delivery, or multiple or repeated deliveries, or a control delivery therapeutic of any of the individual components of the present invention or in combination. Such terms are further meant to include modes of deliveries such as locally, systemically, intravascularly, intramuscularly, intra-peritoneally, inside the blood-brain barrier, organ-specific interventional injection or via other various routes.

“Pharmaceutically acceptable” or “therapeutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Active compound” or “active ingredient” refers to the pharmaceutically active component of the drug which produces the therapeutic effect. In some embodiments, this refers to dexamethasone.

“Subject” refers to a human. The term does not denote a particular age or sex. Thus, adult and newborn subjects, as well as fetuses, whether male or female, are intended to be covered.

“Patient” refers to a subject afflicted with a disease or disorder.

“Reference level” refers to a range of values in a laboratory test or assay result that is considered normal or observed in a healthy population.

“Threshold” refers to a value in a laboratory test or assay result above which is considered an abnormal result. In some embodiments, this abnormal result identifies a patient as having autism.

“Extracellular vesicles” (EVs) refers to secreted membrane-bound vesicles which mediate intercellular communication by transferring biomolecules such as proteins. EVs are secreted into the extracellular space by all cell types and can be found in bodily fluids (e.g. plasma, saliva, semen, urine, cerebral spinal fluid).

“Isolated extracellular vesicles” refers to extracellular vesicles which have been extracted and purified from patient bodily fluid samples, including blood plasma.

“ASD-EVs” refers to EVs isolated from a subject having ASD or suspected of having ASD.

“HC-EVs” refers to EVs isolated from a healthy control subject.

“Healthy control” refers to a patient without ASD, from age- and sex-matched donors.

### Pharmaceutical Compositions

“Pharmaceutical composition” refers to a formulation containing the therapeutically active agents described herein in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (ie. dexamethasone) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. In a preferred embodiment, the active ingredients are mixed under sterile conditions with a pharmaceutically acceptable carrier, and with any preservatives, buffers, or propellants that are required.

“Pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a lotion, cream, ointment or spray applied to the skin). Dexamethasone may also be formulated for inhalation. In certain embodiments, dexamethasone may be simply dissolved or suspended in sterile water. Details of appropriate routes of administration and compositions suitable for same can be found in, for example, U.S. Pat. Nos. 6,110,973; 5,763,493; 5,731,000; 5,541,231; 5,427,798; 5,358,970; and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of dexamethasone which produces a therapeutic effect.

Methods of preparing these formulations or compositions include the step of bringing into association an active compound, such as dexamethasone, with the carrier and, optionally, one or more accessory ingredients. In general, the formulations are prepared by uniformly and intimately bringing into association dexamethasone with liquid carriers, or finely divided solid carriers, or both, and then, if necessary, shaping the product.

Formulations of dexamethasone suitable for oral administration can be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of dexamethasone as an active ingredient. Compositions may also be administered as a bolus, electuary or paste.

To prepare solid dosage forms for oral administration (capsules (including sprinkle capsules and gelatin capsules), tablets, pills, dragées, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragées, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyimide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of dexamethasone to the body. Such dosage forms can be made by dissolving or dispersing the dexamethasone in the proper medium. Absorption enhancers can also be used to increase the flux of the compound across the skin. The rate of such flux can be controlled by either providing a rate controlling membrane or dispersing the compound in a polymer matrix or gel.

“Parenteral administration” and “administered parenterally” refers to modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of dexamethasone include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of dexamethasone include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of dexamethasone in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician having ordinary skill in the art can readily determine and prescribe the therapeutically effective amount of the pharmaceutical composition required. For example, the physician could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with dexamethasone. A larger total dose can be delivered by multiple administrations of the agent. Methods to determine efficacy and dosage are known to those skilled in the art.

In general, a suitable daily dose of dexamethasone used in the methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, dexamethasone may be administered two or three times daily. In other embodiments, dexamethasone will be administered once daily.

The present disclosure includes the use of pharmaceutically acceptable salts of dexamethasone. In certain embodiments, contemplated salts of dexamethasone include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of dexamethasone include, but are not limited to, Na, Ca, K, Mg, Zn or other metal salts. In certain embodiments, contemplated salts of dexamethasone include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXAMPLES

### Example 1

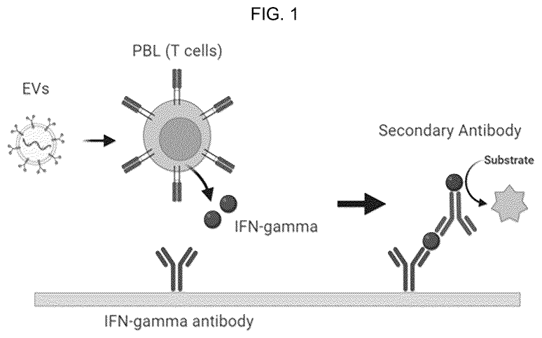

Regulatory role of ASD EVs in inflammation. To explore the potential regulatory role of ASD EVs in inflammation, IFN-gamma secretion from T cells was examined in response to EVs by using an IFN-gamma ELISpot assay (FIGS. 1-4).

Preparation of Peripheral blood lymphocytes (PBL). PBLs comprising approximately 80% T cells were isolated from healthy donors and were grown in plates. The non-adherent PBLs were collected from the supernatant and treated with low-dose recombinant human IL-2 (50 U/mL; eBioscience) to activate.

Preparation of EVs. EVs were isolated from the blood plasma of healthy or ASD subjects used intact (without lysis).

ELISpot assay. The ELISpot assay kit operates on the principles of a modified sandwich ELISA. ELISpot strips are precoated with a primary “capture” antibody which can detect IFN-gamma (FIG. 1). Then, PBLs were incubated with EVs overnight at a ratio of 1:100 (PBL to EVs) on the ELISpot strips using the commercial Human IFN-gamma ELISpot PLUS kit (HRP, Mabtech) prepared according to the manufacturer protocol. IFN-gamma secreted from the PBLs thus binds to the primary “capture” IFN-gamma specific antibody during the incubation period.

To detect the IFN-gamma spots after incubation, a secondary “detection” antibody specific for IFN-gamma and conjugated with biotin is applied to the strip. This binds to IFN-gamma. As a result, the secreted IFN-gamma is immobilized to the strip by the primary capture antibody and further bound by the secondary detection antibody. IFN-gamma is thus trapped between the detection and capture antibodies, much like a “sandwiched”. A streptavidin-conjugated alkaline phosphatase enzyme is then applied, which binds to the biotin of the secondary detection antibody. Last, the substrate solution is added, enabling signal visualization and quantification of IFN-gamma spots.

Results. IFN-gamma ELISpot assay detected IFN-gamma released from T cells in response to EVs (FIG. 1). Although EVs contained IFN-gamma, intact EVs alone without lysis did not cause an IFN-gamma response, suggesting encapsulation of IFN-gamma within EVs (FIG. 2). When PBL was incubated with EVs, healthy control (HC) EVs induced IFN-gamma secretion from PBL. However, ASD EVs stimulated T cells to release elevated levels of IFN-gamma (FIG. 2), demonstrating promotion of the inflammatory response.

Use of dexamethasone for treating ASD. Given that ASD EVs potentiate an inflammatory response by increasing IFN-gamma secretion, the efficacy of dexamethasone, an anti-inflammatory medication, was evaluated in mitigating IFN-gamma secretion induced by ASD EVs. It was observed that 100 nM dexamethasone effectively reduced IFN-gamma secretion triggered by ASD EVs (FIG. 3), demonstrating that dexamethasone can be a potential therapeutic for ASD through inhibiting chronic inflammation and mitigating associated ASD symptoms.

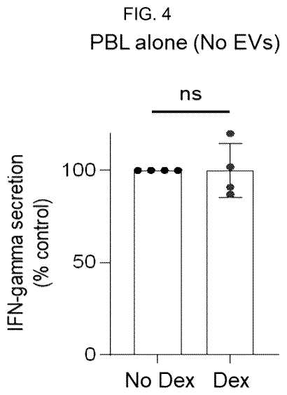

Dexamethasone had little effect on basal IFN-gamma secretion from PBL without stimulus with EVs (No EVs, FIG. 3, and FIG. 4).

Taken together, this demonstrates that dexamethasone specifically inhibits EV-dependent IFN-gamma secretion. Accordingly, dexamethasone can be a potential therapeutic for ASD by inhibiting ASD-EV-mediated IFN-gamma secretion and chronic inflammation.

### Example 2

A 12-year-old male patient has blood drawn for an ASD diagnostic.

Extracellular vesicles are isolated from the plasma fraction using ultracentrifugation. The extracellular vesicles are incubated with commercially available peripheral blood lymphocyte primary cells and secreted IFN-gamma is quantified with an ELISA and compared to a threshold determined by a cohort of healthy patients. The patient is determined to have elevated secreted IFN-gamma in the assay and identified to have autism.

### Example 3

A 6-year-old female patient has blood drawn for an ASD diagnostic.

Extracellular vesicles are isolated from the plasma fraction using size exclusion chromatography. The extracellular vesicles are incubated with donated peripheral blood lymphocyte primary cells and secreted IFN-gamma is quantified with an IFN-gamma ELISpot assay compared to a threshold determined by a cohort of healthy control patients. The patient is determined to have elevated secreted IFN-gamma in the assay and identified to have autism.

### Example 4

A 11-year-old female patient has blood drawn for an ASD diagnostic. Blood is drawn and extracellular vesicles are isolated from the plasma fraction using size exclusion chromatography. The extracellular vesicles are incubated with donated peripheral blood lymphocyte primary cells and secreted IFN-gamma is quantified with an IFN-gamma ELISpot assay compared to threshold determined by a cohort of healthy control patients. The patient is determined to have elevated secreted IFN-gamma in the assay and identified to have autism.

### Example 5

A 22-year-old female who is about 6 months pregnant has amniotic fluid drawn for an ASD prenatal diagnostic. Fetal extracellular vesicles are isolated from the amniotic fluid. The extracellular vesicles are incubated with commercially available peripheral blood lymphocyte primary cells and secreted IFN-gamma is quantified with an IFN-gamma ELISpot assay and compared to threshold determined by a cohort of healthy control patients. The fetus is determined to have elevated secreted IFN-gamma in the assay and susceptibility to developing autism is predicted. Once the infant is born, treatment with therapeutic amounts of anti-inflammatory dexamethasone administered orally may promote improved neurodevelopment and reduce symptom severity in childhood.

### Example 6

A 5-year-old male child is diagnosed with autism. The patient is prescribed an oral dosage of 2 mg of dexamethasone daily to improve neurodevelopment and potentially reduce the autism symptom severity.

### Example 7

A 7-year-old male patient is suspected of having autism. The patient has extracellular vesicles isolated from his plasma and extracellular vesicles are incubated with commercially available peripheral blood lymphocyte primary cells. Secreted IFN-gamma is quantified with an IFN-gamma ELISpot assay. The patient's sample is tested, and the result is 1221 SFU per 106 peripheral blood lymphocyte primary cells. This value is compared to a threshold of 700 SFU per 106 peripheral blood lymphocyte primary cells, which is one standard deviation greater than the mean of a cohort of healthy control subjects. The patient value is above the threshold and the patient is identified to have autism.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of identifying a subject as having autism spectrum disorder (ASD), the method comprising:
identifying an elevated IFN-gamma secretion from the subject, wherein the elevated IFN-gamma secretion is from peripheral blood lymphocytes cells contacted with isolated extracellular vesicles.

2. The method of claim 1, wherein the isolated extracellular vesicles are obtained from a plasma blood sample of the subject.

3. The method of claim 1, wherein the elevated IFN-gamma secretion is compared to the IFN-gamma secretion from a healthy subject.

4. The method of claim 3, wherein the elevated IFN-gamma secretion is increased by at least about 10% relative to the IFN-gamma secretion from the healthy subject.

5. The method of claim 3, wherein the elevated IFN-gamma secretion is greater than a threshold of one standard deviation above the mean of IFN-gamma secretion values from a cohort of healthy subjects.

6. The method of claim 1, wherein the subject is a child.

7. The method of claim 1, wherein the subject is less than 12 years old.

8. The method of claim 1, wherein the subject is in utero.

9. The method of claim 8, wherein the subject in utero is identified with susceptibility to developing autism.

10. The method of claim 1, wherein IFN-gamma secretion is measured using a technique selected from enzyme-linked immunosorbent assay (ELISA), enzyme-linked immunospot (ELISpot) assay, western blot, or a combination thereof.

11. A method of treating ASD in a subject comprising:
administering a therapeutically effective amount of dexamethasone to the subject.

12. The method of claim 11, wherein the administering is via injection.

13. The method of claim 11, wherein the administering is via oral dosage.

14. The method of claim 11, wherein the administering is once daily.

15. The method of claim 11, wherein the therapeutically effective amount is no more than 50 mg.

16. The method of claim 11, wherein the subject is identified as having ASD by identifying an elevated IFN-gamma secretion.

17. The method of claim 16, wherein the elevated IFN-gamma secretion is from peripheral blood lymphocytes cells contacted with isolated extracellular vesicles.
